(19) United States
(12) Patent Application Publication
(10) Pub. No.: US 2024/0325195 A1
(43) Pub. Date: Oct. 3, 2024

(54) CRYOTHERAPEUTIC TREATMENT DEVICES

(71) Applicant: Medtronic Ireland Manufacturing Unlimited Company, Dublin (IE)

(72) Inventors: Carlos H. Lima, Santa Rosa, CA (US); Brian J. Castelli, Santa Rosa, CA (US); Manoj Kumar Singh, Santa Rosa, CA (US)

(21) Appl. No.: 18/577,342

(22) PCT Filed: Aug. 26, 2022
(86) PCT No.: PCT/EP2022/073822

§ 371 Date: Jan. 8, 2024

Related U.S. Application Data
(60) Provisional application No. 63/238,951, filed on Aug. 31, 2021.

(51) Int. Cl. A61F 7/00; A61F 7/12

(52) U.S. Cl. CPC A61F 7/0085; A61F 7/12; A61F 2007/0056; A61F 2007/126

(57) ABSTRACT

A medical device including a proximal end portion and a distal end portion opposite the proximal end portion. An elongate shaft is disposed between the proximal and distal end portions and defines a lumen therebetween. The distal end portion includes a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen.

### FIELD

The present technology is generally related to cryogenic medical devices.

### BACKGROUND

Cryotherapy is a useful treatment modality for many types of medical procedures. In some cases, it is desirable to administer cryotherapy from within a patient's body, such as from within a body lumen. Internal administration of cryotherapy can be advantageous, for example, in at least some neuromodulation procedures. These procedures can include percutaneously introducing a cryotherapeutic element into a patient and then advancing a catheter shaft carrying the cryotherapeutic element along an intravascular path to a suitable treatment location. Once positioned at the treatment location, the cryotherapeutic element can be cooled to modulate nearby nerves. The cooling caused by the cryotherapeutic element, for example, can reduce undesirable local or systemic sympathetic neural activity and thereby achieve various therapeutic benefits.

### SUMMARY

The techniques of this disclosure generally relate to cryogenic medical devices.

Some aspects of the present disclosure involve the recognition that, in cryogenic catheters that include a balloon, inflation of the balloon may occlude blood flow, and that occluding the renal artery may be undesirable. For example, occluding the renal artery could lead to a temporary increase in blood pressure, among other effects. Some aspects of the present disclosure relate to cryogenic catheters that allow blood flow while applying a cryotherapeutic treatment.

In one aspect, the present disclosure provides a medical device including a proximal end portion and a distal end portion opposite the proximal end portion. An elongate shaft is disposed between the proximal and distal end portions and defines a lumen therebetween. The distal end portion includes a plurality of tubes extending from a distal end of the elongate shaft, the plurality of tubes define a fluid pathway therein in communication with the lumen for circulation of cryogenic fluid. The plurality of tubes have a common exhaust lumen in fluid communication with the lumen.

In another aspect of this embodiment, the device further includes at least one thermally conductive treatment element disposed about at least one of the plurality of tubes.

In another aspect of this embodiment, the at least one thermally conductive treatment elements include a plurality of thermally conductive treatment elements longitudinally spaced along at least one of the plurality of tubes.

In another aspect of this embodiment, the plurality of thermally conductive treatment elements is circumferentially disposed about at least one of the plurality of tubes.

In another aspect of this embodiment, the plurality of tubes defines a basket.

In another aspect of this embodiment, the plurality of tubes surrounds the exhaust lumen.

In another aspect of this embodiment, each of the plurality of tubes defines a prong.

In another aspect of this embodiment, the plurality of prongs is transitionable from a first substantially linear configuration to a second expanded configuration, each of the plurality of prongs are curved in different directions from each and every other one of the plurality or prongs when in the second expanded configuration.

In another aspect of this embodiment, each prong of the plurality of prongs is biased to extend outwards radially when in the second expanded configuration.

In another aspect of this embodiment, the entirety of the prongs is thermally conductive.

In one aspect, a medical device includes a proximal end portion and a distal end portion opposite the proximal end portion. An elongate shaft is disposed between the proximal and distal end portions and defines a lumen therebetween. The distal end portion includes a plurality of tubes extending from a distal end of the elongate shaft. The plurality of tubes define a fluid pathway therein in communication with the lumen for circulation of cryogenic fluid. The plurality of tubes having a common exhaust lumen in fluid communication with the lumen and the plurality of tubes circumferentially surround the exhaust lumen.

In another aspect of this embodiment, the exhaust lumen is co-axial with the elongate shaft.

In another aspect of this embodiment, the plurality of tubes defines a basket.

In another aspect of this embodiment, the device further includes at least one thermally conductive treatment element disposed about at least one of the plurality of tubes.

In another aspect of this embodiment, the at least one thermally conductive treatment elements include a plurality of thermally conductive treatment elements longitudinally spaced along at least one of the plurality of tubes.

In another aspect of this embodiment, the plurality of thermally conductive treatment elements is circumferentially disposed about at least one of the plurality of tubes.

In another aspect of this embodiment, the plurality of tubes is composed of Nitinol.

In another aspect of this embodiment, each of the plurality of tubes includes at least two of the thermally conductive elements.

In another aspect of this embodiment, the thermally conductive elements are composed of gold.

In one aspect, a medical device includes a proximal end portion and a distal end portion opposite the proximal end portion. An elongate shaft is disposed between the proximal and distal end portions and defines a lumen therebetween. The distal end portion includes a plurality of tubes extends from a distal end of the elongate shaft. The plurality of tubes define a fluid pathway therein in communication with the lumen for circulation of cryogenic fluid. The plurality of tubes have a common exhaust lumen in fluid communication with the lumen and the plurality of tubes circumferentially surround the exhaust lumen and define a basket when expanded. The plurality of tubes is transitionable from a first position in which the tubes are disposed within the elongate shaft to a second position in which the tubes extend outward and away from the elongate shaft. A plurality of thermally conductive elements is circumferentially disposed about each of the plurality of tubes.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a system view of a cryogenic device and console constructed in accordance with the principles of the present application;

FIG. 2 is a cross-sectional view across A-A shown in FIG. 1;

FIG. 3 is a cross-sectional view across B-B shown in FIG. 1;

FIG. 4 is a cross-sectional view of another embodiment of a cryogenic device in a first position;

FIG. 5A is a side view of the embodiment of the medical device shown in FIG. 4 in a second position; and

FIG. 5B is a cross-sectional view of the embodiment of the medical device shown FIG. 5A.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

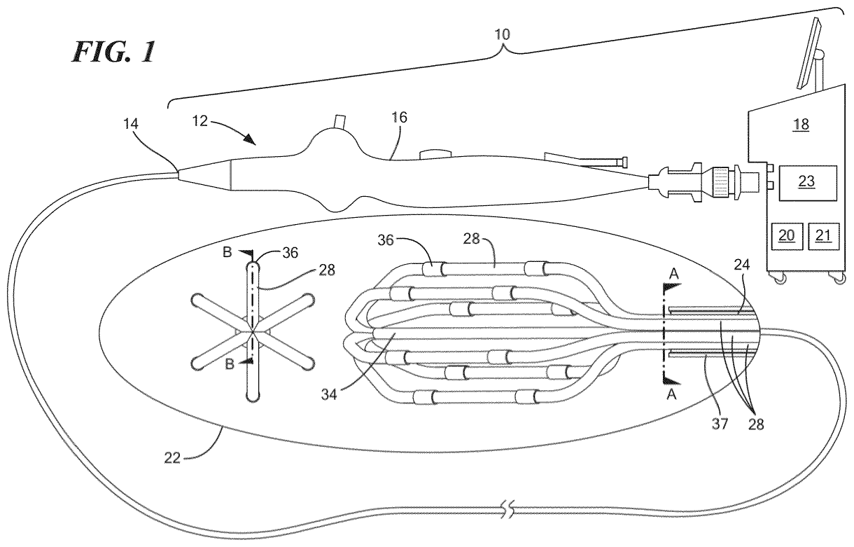

Referring now to the drawings in which like reference designators refer to like elements, there is shown in FIG. 1 an exemplary medical system constructed in accordance with the principles of the present application and designated generally as “10.” The system may include a medical device 12 including a proximal end portion 14 in fluid communication with a handle 16. The handle 16 can also include connectors that are matable directly to a cryogenic fluid supply/exhaust and console or indirectly by way of one or more umbilicals (not shown). In the exemplary system, the fluid supply and exhaust, as well as various control mechanisms for the system are housed within a single control unit or console 18. In one embodiment, the console 18 may include a refrigerant source 20 or fluid supply reservoir, an exhaust chamber 21, and processing circuitry 23 having a processor and memory configured to monitor and control the delivery and/or exhaust of refrigerant from the medical device 12. The console 18 is configured to deliver refrigerant, such as nitrous oxide, which passes through the internal piping of the console 18 before being transferred to the medical device 12 via the umbilicals. As the refrigerant is released in the medical device 12 under vacuum, the temperature of the fluid drops because of the positive Joule-Thomson coefficient of nitrous oxide. Then the liquid evaporates as it absorbs heat from the medical device 12 which results in the freezing of portions of the device 12. The refrigerant vapor is then returned through the vacuum path via the umbilical and into the console 18, where it may be evacuated through a scavenging line or through the exhaust chamber 21. In addition to providing an exhaust function for the fluid supply of the medical device 12, the console 18 can also recover and/or recirculate the cooling fluid. As such, the system 10 may be referred to herein as a closed-loop system.

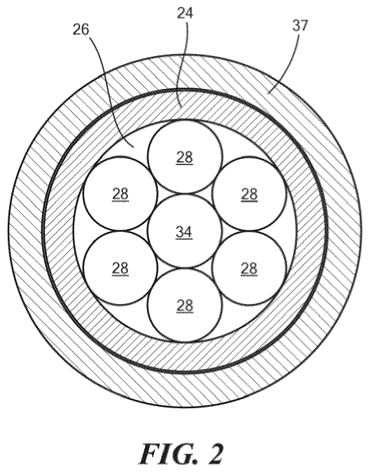

Referring now to FIGS. 1-3, in some configurations, the medical device 12 disclosed herein may be delivered through a blood vessel to a target tissue region through a guide wire or guide catheter 37, which is configured to be advanced through the blood vessels of a patient. The medical device 12 may further include a distal end portion 22 about which cryogenic energy is exchanged between the medical device 12 and a target tissue, for example, renal tissue. For example, the delivery and/or circulation of cryogenic fluid or refrigerant within the distal end portion 22 cools at least a portion of the distal end portion 22 to a temperature low enough to extract heat from, and thus cryoablate, the target tissue. The medical device 12 may include an elongate shaft 24 disposed between the proximal end portion 14 and the distal end portion 22 and which further defines a lumen 26 (shown in FIG. 2) there through. The elongate shaft 24 may be at least partially disposed or received within the sheath or guide catheter 37 defining an opening at a distal end through which a plurality of tubes 28 may extend from the elongate shaft 24. The plurality of tubes 28 may be composed of a preformed flexible material, for example Nitinol, that when advanced beyond the distal end of the guide catheter in which the tubes 28 are contained, expands into a shape that conforms with the blood vessel and can be retracted, while elongating, to fit within the lumen, and are configured to transport cryogenic fluid or refrigerant. Additionally and/or alternatively, the tubes 28 may also expand into the shape that conforms with the blood vessel when the guide catheter 37 is retracted towards the proximal end portion 14 with respect to the elongate shaft 24. In some configurations, the plurality of tubes 28 are curved or arcuate in shape and are transitional from a first configuration in which they are disposed within the shaft 24 to a second position in which they extend outward away from the shaft 24. In some configurations, the plurality of tubes 28 are slidable with respect to the elongate shaft 24 and in other configurations, the plurality of tubes 28 may be fixed to the elongate shaft 24. For example, in some configurations, each of the plurality of tubes 28 may be independently movable with respect to each and every other tubes 28 to provide for particular ablation patterns. In other configurations, the plurality of tubes 28 includes six tubes that define a basket configuration as shown in FIG. 1. The plurality of tubes 28 are configured to merge together at a common point such that fluid circulating within each respective tube 28 comingles with fluid within each respective tube 28. In one configuration, each of the plurality of tubes 28 join together at a distal end portion of each respective tube 28 such that each of the plurality of tubes 28 are in fluid communication with each and every other one of the plurality of tubes 28.

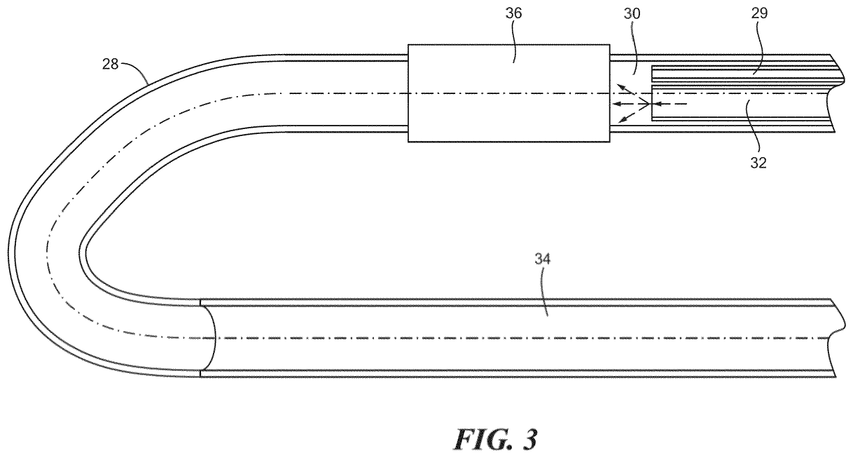

Continuing to refer to FIG. 3, the plurality of tubes 28 defines a fluid pathway 30 therein in communication with the lumen 26 for circulation of cryogenic fluid. For example, disposed within each of the plurality of tubes 28 may be an inflow tube 32 composed of, for example, polyimide, configured to circulate cryogenic fluid within the plurality of tubes 28 and a thermocouple wire 29 configured to measure a temperature within the plurality of tubes 28. The plurality of tubes 28 each have a common exhaust lumen 34 in fluid communication with the lumen 26 and with the fluid pathway 30. For example, as shown in FIGS. 1-3, the plurality of tubes 28 merge into a common exhaust lumen 34, which returns the cryogenic fluid within back through the lumen 26. In the configuration shown in FIGS. 1-3, the exhaust lumen 34 is coaxial with the major longitudinal axis of the lumen 26, however, in other configurations the exhaust lumen 34 may be offset from the major longitudinal axis of the lumen 26. In some configurations, the plurality of tubes 28 circumscribe the exhaust lumen 34, for example, as shown in FIGS. 1-3. In the configuration shown in FIGS. 1-3, the exhaust lumen is parallel to at least a portion of the plurality of tubes 28. A vacuum pump in the console 18 creates a low pressure environment in one or more lumens within the elongate shaft 24 so that refrigerant is drawn into the lumen(s), away from the plurality of tubes 28, towards the proximal end 14 of the elongate shaft 24, and into the exhaust chamber 21 within the console 18.

Continuing to refer to FIGS. 1-3, at least one thermally conductive element 36 may be disposed about at least a portion of at least one of the plurality of tubes 28. The at least one thermally conductive element 36 may be, for example, an annular element such as a thermally conductive band or ring element circumscribing a portion of the respective one of the plurality of tubes 28. In one configuration, the at least one thermally conductive element 36 is composed of gold, but may be composed of other highly thermally conductive metals or metal alloys such as stainless steel, titanium, or the like. The at least one thermally conductive element 36 provides a contact surface at which cryogenic energy may be exchanged between the device 10 and the target tissue. In the configuration shown in FIGS. 1-2, each of the plurality of tubes 28 includes a plurality of the at least one thermally conductive element 36 longitudinal spaced along each tube 28. The plurality of the at least one thermally conductive element 36 may be radially aligned with corresponding thermally conductive elements 36 on an adjacent tube 28 such that when the basket is expanded, or otherwise deployed, the plurality of the at least one thermally conductive element 36 defines a circumferential contact surface to create a circumferential conduction block within, for example, the renal artery.

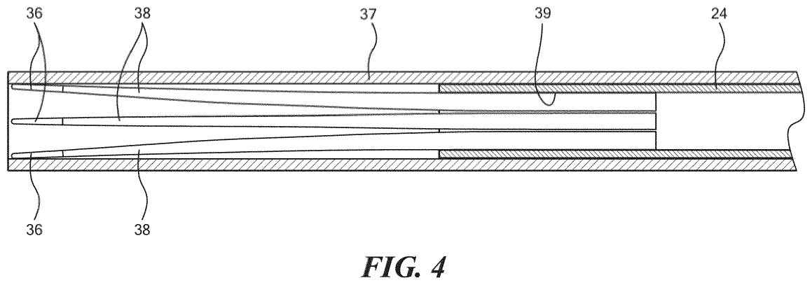

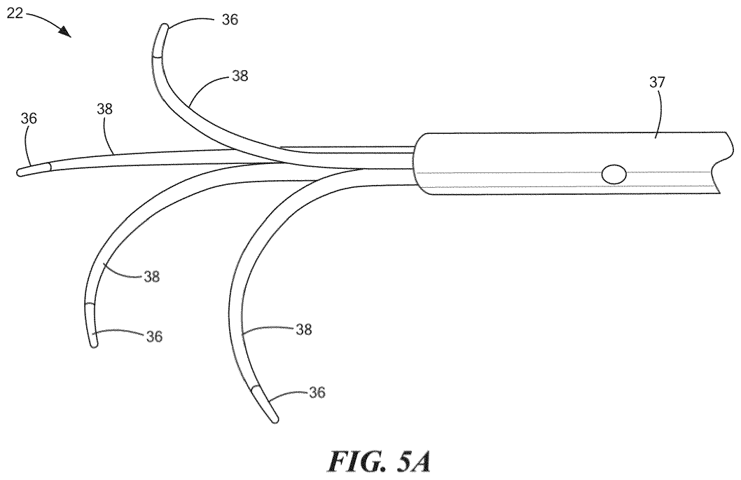

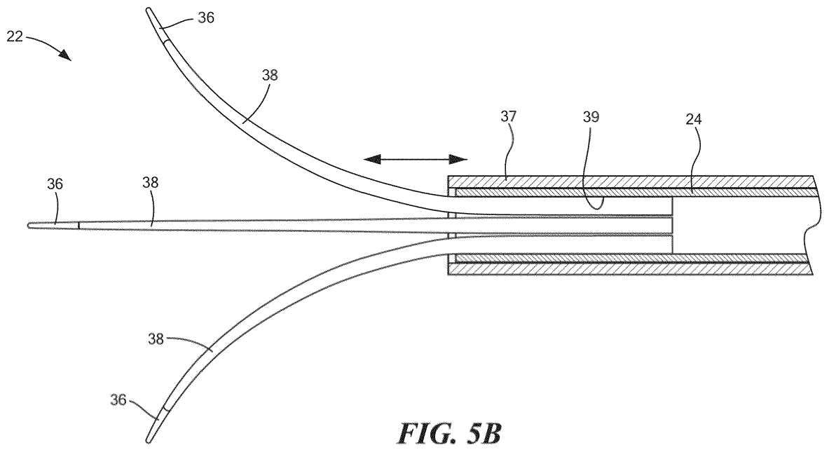

FIG. 4-5B illustrate another embodiment of a medical device 12 may be advanced through a sheath or the guide catheter 37. The guide catheter 37 may be movable with respect to the elongate shaft 24 in longitudinal and/or rotational directions. That is, the guide catheter 37 may be slidably and/or rotatably moveable with respect to the elongate shaft 24. The elongate shaft 24 may further define a lumen therein for the introduction of a plurality of prongs 38, which is either coupled to an inner wall 39 of the elongate shaft lumen, or the distal end of the elongate shaft 24. Each prong 38 may contain a preformed part of a flexible material, such as Nitinol, and may have a natural bias to extend outward radially. The longitudinal movement or retraction of the guide catheter 37 towards the proximal end portion 14 may result in the transitioning of the plurality of prongs 38 from a first substantially linear configuration to a second expanded configuration due to the lack of resistance against the biased prongs 38. Additionally and/or alternatively, the longitudinal movement or advancement of the distal end portion 22 beyond the distal end of the guide catheter 37 may also result in the transitioning of the plurality of prongs 38 from the first substantially linear configuration to the second expanded configuration.

Continuing to refer to FIGS. 4-5B, the plurality of prongs 38 may extend from the elongate shaft 24. The prongs 38 can have a blunted end so as not to penetrate tissue when contact is made between the target tissue and prongs 38. In some configurations, each prong 38 and/or thermally conductive element 36 of the plurality of prongs 38 tapers in width as it extends distally and, in some such configurations, may have a blunted pointed edge so as not to penetrate tissue when contact is made between the target tissue and prongs 38. Additionally and/or alternatively, in other configurations, each prong 38 and/or conductive element 36 may have a uniform diameter throughout its entirety and, in some such configurations, each prong of the plurality of prongs 38 may lack a pointed edge. As shown in FIG. 4, each prong 38 may transition from a straightened or first substantially linear configuration to a second expanded configuration (as shown in FIGS. 5A-5B) once the guide catheter 37 is retracted towards the proximal end portion 14 of the device 12. In other words, the plurality of prongs 38 is in the first substantially linear configuration when fully disposed within the guide catheter 37 and transitions to the second expanded configuration once the guide catheter 37 is retracted towards the proximal end portion 14, thus exposing the plurality of prongs 38 to the ambient environment within the blood vessel. The retraction of the guide catheter 37 results in less resistance being applied or directed towards the prongs 38, which each have a natural bias to expand radially outward when in the second expanded configuration. Thus, the collapsing and/or expansion of the plurality of prongs 38 is controlled by the longitudinal movement of the guide catheter 37, which may also be coupled to the handle 16 of the medical device 12 by at least one pull wire (not shown). The guide catheter 37 may moveable longitudinally by manipulating the pull wires via the medical device handle 16. In the second expanded configuration shown in FIG. 4A, the plurality of prongs defines a diameter larger than a diameter of the distal end of the shaft 24, and each of the prongs 38 is configured to be in contact with an area of target tissue. Following a treatment procedure, the guide catheter 37 may be advanced over the elongate shaft 24 and the plurality of prongs 38. As the guide catheter 37 is advanced over the plurality of prongs 38, the guide catheter applies resistance against each prong 38 which transitions the plurality of prongs 38 back to the first substantially linear configuration so that the prongs 38 may be retracted through the guide catheter 37 and removed from the patient's vasculature.

Further, each prong 38 may be composed of thermally conductive metals and/or metal alloys, for example, stainless steel, titanium, or gold. Disposed within each prong 38 may be the fluid pathway 30 (as shown in FIG. 3) for a continuous flow of cryogenic fluid to the tip of each prong 38. Each of the prongs 38 may be in fluid communication with the common exhaust lumen 34 proximal to the proximal ends of each of the prongs 38. In one configuration, the plurality of prongs 38 each extend outward radially, and away from the shaft 24 in different directions. For example, the plurality of prongs 38 may define a circumferential configuration to create a circumferential lesion. In another configuration, only the distal end of the prongs 38 are thermally conductive, for example, by including thermally conductive element 36, and the remainder of prongs 38 are composed of a non-thermally conductive material.

Although not described in detail herein, it is to be understood that the console 18 may control or regulate the delivery and exhausting of cryogenic fluid (e.g., refrigerant) from each prong 38 independent of the other prongs 38. For example, in one configuration where the plurality of prongs 38 includes four prongs, refrigerant may be delivered to only two, or a first pair, of prongs 38. The controlled delivery and/or exhausting of refrigerant from each prong 38 allows clinicians to more precisely treat a targeted tissue area by providing the ability for clinicians to widen or lessen the treatment radius when treating tissue. Further, it is to be understood that the plurality of prongs 38 may include two or more prongs 38. For example, the plurality of prongs 38 may include any number of prongs 38 such as, but not limited to, three, four, five, six, seven, eight, or nine prongs, etc.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims. The features of various embodiments can be combined to form further embodiments of the invention that may not be explicitly described or illustrated. Each embodiment and each aspect so defined may be combined with any other embodiment or with any other aspect unless clearly indicated to the contrary.

### [1] Aspects and Embodiments of the Invention May be Defined by the Following Clauses.

Clause 1. A medical device, comprising:
a proximal end portion and a distal end portion opposite the proximal end portion;
an elongate shaft disposed between the proximal and distal end portions and a defining a lumen therebetween; and
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen.

Clause 2. The device of Clause 1, further including at least one thermally conductive treatment element disposed about at least one of the plurality of tubes.

Clause 3. The device of Clause 2, wherein the at least one thermally conductive treatment elements include a plurality of thermally conductive treatment elements longitudinally spaced along at least one of the plurality of tubes.

Clause 4. The device of Clause 3, wherein the plurality of thermally conductive treatment elements is circumferentially disposed about at least one of the plurality of tubes.

Clause 5. The device of Clause 1, wherein the plurality of tubes defines a basket.

Clause 6. The device of Clause 5, wherein the plurality of tubes surrounds the exhaust lumen.

Clause 7. The device of Clause 1, wherein each of the plurality of tubes defines a prong.

Clause 8. The device of Clause 7, wherein the plurality of prongs is transitionable from a first substantially linear configuration to a second expanded configuration, each of the plurality of prongs are curved in different directions from each and every other one of the plurality of prongs when in the second expanded configuration.

Clause 9. The device of Clause 8, wherein each prong of the plurality of prongs is biased to extend outwards radially when in the second expanded configuration.

Clause 10. The device of Clause 9, wherein the entirety of the prongs is thermally conductive.

Clause 11. A medical device, comprising:
a proximal end portion and a distal end portion opposite the proximal end portion;
an elongate shaft disposed between the proximal and distal end portions and defining a lumen therebetween; and
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen and the plurality of tubes circumferentially surround the exhaust lumen.

Clause 12. The device of Clause 11, wherein the exhaust lumen is co-axial with the elongate shaft.

Clause 13. The device of Clause 11, wherein the plurality of tubes defines a basket.

Clause 14. The device of Clause 11, further including at least one thermally conductive treatment element disposed about at least one of the plurality of tubes.

Clause 15. The device of Clause 14, wherein the at least one thermally conductive treatment elements include a plurality of thermally conductive treatment elements longitudinally spaced along at least one of the plurality of tubes.

Clause 16. The device of Clause 15, wherein the plurality of thermally conductive treatment elements is circumferentially disposed about at least one of the plurality of tubes.

Clause 17. The device of Clause 16, wherein the plurality of tubes is composed of Nitinol.

Clause 18. The device of Clause 17, wherein each of the plurality of tubes includes at least two of the thermally conductive elements.

Clause 19. The device of Clause 18, wherein the thermally conductive elements are composed of gold.

Clause 20. A medical device, comprising:
a proximal end portion and a distal end portion opposite the proximal end portion;
an elongate shaft disposed between the proximal and distal end portions and defining a lumen therebetween;
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen and the plurality of tubes circumferentially surround the exhaust lumen and define a basket when expanded, the plurality of tubes being transitionable from a first position in which the tubes are disposed within the elongate shaft to a second position in which the tubes extend outward and away from the elongate shaft; and
a plurality of thermally conductive elements circumferentially disposed about each of the plurality of tubes.

1. A medical device, comprising:
a proximal end portion and a distal end portion opposite the proximal end portion;
an elongate shaft disposed between the proximal and distal end portions and a defining a lumen therebetween; and
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen.

2. The device of claim 1, further including at least one thermally conductive treatment element disposed about at least one of the plurality of tubes.

3. The device of claim 2, wherein the at least one thermally conductive treatment elements include a plurality of thermally conductive treatment elements longitudinally spaced along at least one of the plurality of tubes.

4. The device of claim 3, wherein the plurality of thermally conductive treatment elements is circumferentially disposed about at least one of the plurality of tubes.

5. The device of claim 1, wherein the plurality of tubes defines a basket.

6. The device of claim 1, wherein the plurality of tubes surrounds the exhaust lumen.

7. The device of claim 1, wherein each of the plurality of tubes defines a prong.

8. The device of claim 7, wherein the plurality of prongs is transitionable from a first substantially linear configuration to a second expanded configuration, each of the plurality of prongs are curved in different directions from each and every other one of the plurality of prongs when in the second expanded configuration.

9. The device of claim 8, wherein each prong of the plurality of prongs is biased to extend outwards radially when in the second expanded configuration.

10. The device of claim 7, wherein the entirety of the prongs is thermally conductive.

11. A medical device, comprising:
a proximal end portion and a distal end portion opposite the proximal end portion;
an elongate shaft disposed between the proximal and distal end portions and defining a lumen therebetween; and
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen and the plurality of tubes circumferentially surround the exhaust lumen.

12. The device of claim 11, wherein the exhaust lumen is co-axial with the elongate shaft.

13. The device of claim 11, wherein the plurality of tubes defines a basket.

14. The device of claim 11, further including at least one thermally conductive treatment element disposed about at least one of the plurality of tubes.

15. The device of claim 14, wherein the at least one thermally conductive treatment elements include a plurality of thermally conductive treatment elements longitudinally spaced along at least one of the plurality of tubes.

16. The device of claim 14, wherein the plurality of thermally conductive treatment elements is circumferentially disposed about at least one of the plurality of tubes.

17. The device of claim 11, wherein the plurality of tubes is composed of Nitinol.

18. The device of claim 14, wherein each of the plurality of tubes includes at least two of the thermally conductive elements; and/or wherein the thermally conductive elements are composed of gold.

19. A medical device, comprising:
a proximal end portion and a distal end portion opposite the proximal end portion;
an elongate shaft disposed between the proximal and distal end portions and defining a lumen there between;
the distal end portion including a plurality of tubes extending from a distal end of the elongate shaft, the plurality of tubes defining a fluid pathway therein in communication with the lumen for circulation of cryogenic fluid, the plurality of tubes having a common exhaust lumen in fluid communication with the lumen and the plurality of tubes circumferentially surround the exhaust lumen and define a basket when expanded, the plurality of tubes being transitionable from a first position in which the tubes are disposed within the elongate shaft to a second position in which the tubes extend outward and away from the elongate shaft; and
a plurality of thermally conductive elements circumferentially disposed about each of the plurality of tubes.
